(19) United States
(12) Patent Application Publication
(10) Pub. No.: US 2026/0094681 A1
(43) Pub. Date: Apr. 2, 2026

(54) CARD AND CARD READERS TO IMPROVE STORAGE AND SHARING OF HEALTHCARE INFORMATION

(71) Applicant: Richard Schriewer, New Braunfels, TX (US)

(72) Inventor: Richard Schriewer, New Braunfels, TX (US)

(21) Appl. No.: 19/342,074

(22) Filed: Sep. 26, 2025

Related U.S. Application Data
(60) Provisional application No. 63/700,664, filed on Sep. 28, 2024.

(51) Int. Cl. G16H 10/65

(52) U.S. Cl. CPC G16H 10/65

(57) ABSTRACT

The invention is a system for managing patient medical information, comprising a medical identity card, a mobile reader, and a stationary reader. The card holds various data types, with basic data visibly printed, less sensitive data printed in UV-visible code, and highly sensitive data stored on a chip. The card may also include a reflective surface and a locating device. The mobile reader, carried by healthcare personnel, emits UV light to read the card's code. The stationary reader, typically located at a doctor's office, connects to a computer to read the card's chip or access a remote database. It includes security measures to ensure only authorized individuals can access the information.

### CROSS-REFERENCE TO RELATED APPLICATIONS

This application claims the benefit of, and priority to, US Provisional Patent Application No.: 63/700,654 filed Sep. 28, 2024, titled “CARD AND CARD READERS TO IMPROVE STORAGE AND SHARING OF HEALTHCARE INFORMATION.” This application is herein incorporated by reference in its entirety.

### BACKGROUND

### Field of Art

The present disclosure is related to the field of improving how individuals share their medical data through innovative technological solutions.

### Discussion of the State of the Art

In the field of medical record keeping and patient information management, there are several persistent challenges that have yet to be effectively addressed. These challenges have a direct impact on the efficiency of healthcare delivery, the accuracy of medical information, and the safety of patients.

One of the primary issues is the excessive amount of time it takes to complete new patient medical forms and update existing ones. Furthermore, these systems often lack the necessary interoperability to allow for the seamless sharing of information between different healthcare providers. This process is often laborious and time-consuming, requiring patients to recall and accurately report a multitude of details about their medical history, including medications, surgeries, allergies, family member medical histories and other pertinent health information. This not only places an undue burden on patients, but also on healthcare providers who must then input this information into their systems, which may include medical record software, taking time away from direct patient care and affecting the medical care provider's schedule.

Additionally, many patients struggle with the complexity of medical terminology, making it difficult for them to accurately complete these forms. This can lead to errors or omissions in the patient's medical record, which in turn can compromise the quality of care they receive. The problem is further exacerbated when patients are required to remember specific details such as the names of doctors, medication dosage parameters, and dates of surgeries.

In emergency situations, these challenges become even more critical. Emergency medical professionals often need immediate access to a patient's medical status and history in order to make life-saving decisions. However, in many cases, the patient may be incoherent or unconscious, making it impossible to obtain this information directly from them.

Previous attempts to solve these problems have been largely unsuccessful. For instance, medical alert bracelets, while useful in certain situations, can only convey a limited amount of information and are not always up-to-date. Moreover, they do not address the broader issues related to the management and accessibility of a patient's comprehensive medical history as technologies such as medical alert bracelets tend to be narrowly focused on a particular disease or condition rather than providing a more holistic and comprehensive overview of the individual's medical history.

Manual entry of patient information into computer systems, another common practice, is also fraught with problems. Not only is this process time-consuming, but it is also prone to human error, which can result in inaccurate or incomplete patient records. Additionally, the different healthcare provider systems may have varied templates for data which make it difficult to share within different systems.

In summary, there is a clear and urgent need for a more efficient and reliable method of managing patient information throughout the healthcare sector. The current methods are time-consuming, prone to errors, and fail to provide immediate access to critical medical information in emergency situations. This not only compromises the security and quality of patient care and its data, but also places unnecessary strain on healthcare providers.

### SUMMARY

The present invention provides a novel system for managing patient medical information, designed to address the challenges inherent in existing methods. The system comprises a medical identity card, a mobile reader, and a stationary reader. This invention offers significant benefits in terms of time efficiency, data accuracy, and accessibility of patient medical records.

The medical identity card is designed with various features to enhance its functionality and ease of use. It may have a reflective surface to facilitate easy location. The card contains different types of patient data, stored using various methods based on the sensitivity of the information. Basic, non-medical data is printed using easily readable ink. Less sensitive medical data, such as allergies, is encoded and printed using ink that is only visible under UV light. Highly sensitive medical data, such as medical history, is stored on a chip that requires a specialized reader to access the information. Additionally the card may comprise a data chip that provides access to a remote database which comprises patient medical data and history. The data may be written to the chip before or after assembly into the card.

The mobile reader, designed to be carried by healthcare personnel, is equipped with UV light emission capabilities to read the encoded information on the identity card. This feature is particularly beneficial in emergency situations, where quick access to patient information is critical.

The stationary reader, designed for use at a doctor's office, can connect to a computer and read the chip on the identity card. It can either extract information directly from the card or access it from a remote database. The stationary reader is equipped with high-level security measures to ensure that only authorized personnel, such as doctors, can access the sensitive medical information.

This invention overcomes the challenges of prior solutions by significantly reducing the time and effort required by patients and healthcare professionals to manage medical records. The system ensures that the information is kept up-to-date by medical personnel as care is administered, resulting in accurate and easily accessible medical records. By pairing with a healthcare computer, the notes, billing and coding information may be shared between the card, database, and medical systems. The card also allows emergency medical personnel to access relevant medical information quickly and in more detail than a medical alert bracelet.

The key elements of the invention include the Patient Identity Card, Mobile Reader, Stationary Reader, and Database. The card may feature a reflective surface, a chip, an easily readable information box, a UV light code, and a locating device. This invention offers a convenient, efficient, and secure solution for transferring and updating medical records, thereby enhancing the quality of patient care.

### BRIEF DESCRIPTION OF THE DRAWING FIGURES

The accompanying drawings illustrate several embodiments and, together with the description, serve to explain the principles of the invention according to the embodiments. It will be appreciated by one skilled in the art that the particular arrangements illustrated in the drawings are merely exemplary and are not to be considered as limiting of the scope of the invention or the claims herein in any way.

FIG. 1 illustrates a network diagram of an embodiment of the disclosed invention.

FIG. 2a-2c illustrate a diagram of the identity card in an embodiment of the disclosed invention.

FIG. 3 illustrates a diagram of the identity card in an embodiment of the disclosed invention.

FIG. 3 illustrates a diagram of the mobile reader in an embodiment of the disclosed invention.

FIG. 4 illustrates a diagram of the stationary reader in an embodiment of the disclosed invention.

FIG. 5 illustrates a diagram of the processing system in an embodiment of the disclosed invention.

FIG. 6 illustrates one embodiment of the computing architecture that supports an embodiment of the inventive disclosure.

FIG. 7 illustrates components of a system architecture that supports an embodiment of the inventive disclosure.

FIG. 8 illustrates components of a computing device that supports an embodiment of the inventive disclosure.

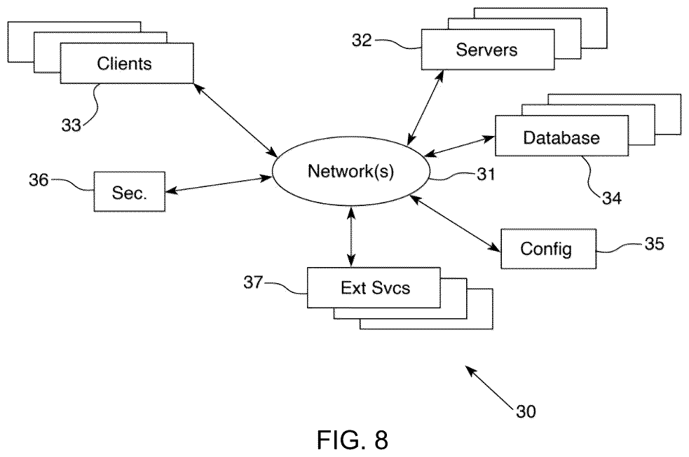

FIG. 9 illustrates components of a computing device that supports an embodiment of the inventive disclosure.

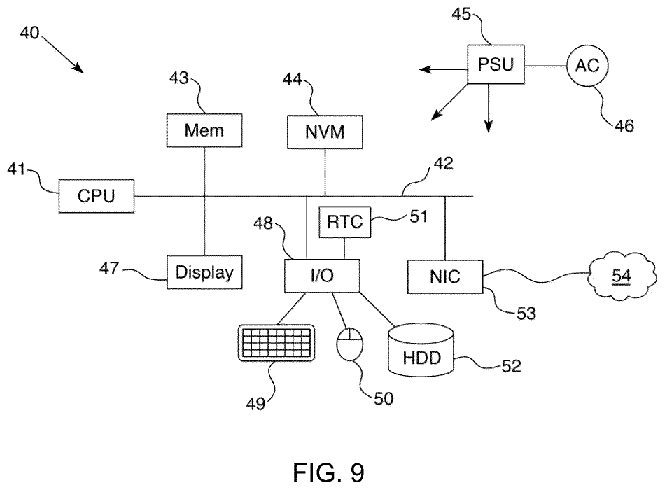

### DETAILED DESCRIPTION OF EMBODIMENTS

The present invention is for a system of Cards and Card Readers designed to enhance the sharing of healthcare information among patients and healthcare professionals. The invention is described by reference to various elements herein. It should be noted, however, that although the various elements of the inventive apparatus are described separately below, the elements need not necessarily be separate. The various embodiments may be interconnected and may be cut out of a singular block or mold. The variety of different ways of forming an inventive apparatus, in accordance with the disclosure herein, may be varied without departing from the scope of the invention.

Generally, one or more different embodiments may be described in the present application. Further, for one or more of the embodiments described herein, numerous alternative arrangements may be described. It should be appreciated that these are presented for illustrative purposes only and are not limiting of the embodiments contained herein or the claims presented herein in any way. One or more of the arrangements may be widely applicable to numerous embodiments, as may be readily apparent from the disclosure. In general, arrangements are described in sufficient detail to enable those skilled in the art to practice one or more of the embodiments, and it should be appreciated that other arrangements may be utilized and that structural changes may be made without departing from the scope of the embodiments. Particular features of one or more of the embodiments described herein may be described with reference to one or more particular embodiments or figures that form a part of the present disclosure, and in which are shown, by way of illustration, specific arrangements of one or more of the aspects. It should be appreciated, however, that such features are not limited to usage in the one or more particular embodiments or figures with reference to which they are described. The present disclosure is neither a literal description of all arrangements of one or more of the embodiments nor a listing of features of one or more of the embodiments that must be present in all arrangements.

Headings of sections provided in this patent application and the title of this patent application are for convenience only and are not to be taken as limiting the disclosure in any way.

Devices and parts that are connected to each other need not be in continuous connection with each other, unless expressly specified otherwise. In addition, devices and parts that are connected with each other may be connected directly or indirectly through one or more connection means or intermediaries.

A description of an aspect with several components in connection with each other does not imply that all such components are required. To the contrary, a variety of optional components may be described to illustrate a wide variety of possible embodiments and in order to more fully illustrate one or more embodiments. Similarly, although process steps, method steps, or the like may be described in a sequential order, such processes and methods may generally be configured to work in alternate orders, unless specifically stated to the contrary. In other words, any sequence or order of steps that may be described in this patent application does not, in and of itself, indicate a requirement that the steps be performed in that order. The steps of described processes may be performed in any order practical. Further, some steps may be performed simultaneously despite being described or implied as occurring non-simultaneously (e.g., because one step is described after the other step). Moreover, the illustration of a process by its depiction in a drawing does not imply that the illustrated process is exclusive of other variations and modifications thereto, does not imply that the illustrated process or any of its steps are necessary to one or more of the embodiments, and does not imply that the illustrated process is preferred. Also, steps are generally described once per aspect, but this does not mean they must occur once, or that they may only occur once each time a process, or method is carried out or executed. Some steps may be omitted in some embodiments or some occurrences, or some steps may be executed more than once in a given aspect or occurrence.

When a single device or article is described herein, it will be readily apparent that more than one device or article may be used in place of a single device or article. Similarly, where more than one device or article is described herein, it will be readily apparent that a single device or article may be used in place of the more than one device or article.

The functionality or the features of a device may be alternatively embodied by one or more other devices that are not explicitly described as having such functionality or features. Thus, other embodiments need not include the device itself.

Techniques and mechanisms described or referenced herein will sometimes be described in singular form for clarity. However, it should be appreciated that particular embodiments may include multiple iterations of a technique or multiple instantiations of a mechanism unless noted otherwise. Alternate implementations are included within the scope of various embodiments in which, for example, functions may be executed out of order from that shown or discussed, including substantially concurrently or in reverse order, depending on the functionality involved, as would be understood by those having ordinary skill in the art.

### System

The system of the present invention is comprised of the following elements illustrated below. The elements individually or in combination provide the benefits described above.

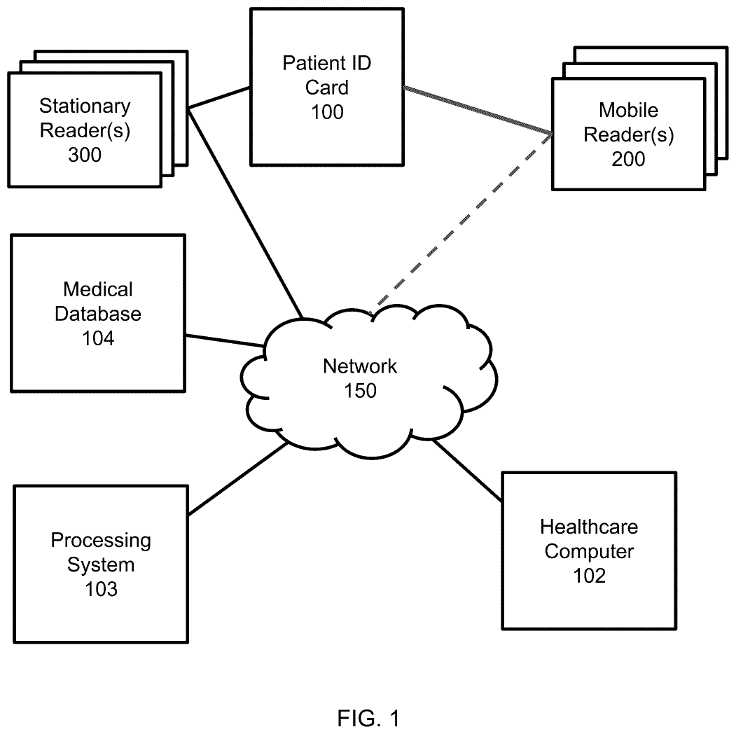

In reference to FIG. 1, the present invention is for a comprehensive system comprising a Patient Identity Card 100, a Mobile Reader 200, a Stationary Reader 300, a processing system 103, a healthcare computer 102, a database 104, and a network 150. The system facilitates access to patient medical records in both emergency and non-emergency incidents.

Patient Identity card 100 may be a generally planar, credit-card-sized device configured to carry and convey patient identification and medical information to human and machine readers. The card 100 may have a length of 3.370 inches with a range of 3 to 3.5 inches, the width may be 2.125 inches with a range of 2 to 2.25 inches, and the thickness may be 0.03 inches with a range of 0.03 to 0.10 inches. These dimensions are exemplary and the size of the card may be adjusted depending on the application as would be apparent to one of ordinary skill in the art. The card may present the information in a plurality of modalities, including visible readable text and images (e.g., patient name, photograph, allergies, medical alert icons), a light-reactive ink region that reveals an emergency subset when illuminated at a selected wavelength, one or more optically scannable codes (e.g., QR, Aztec, 1-D barcode) that encode data fields or locators, and one or more embedded data chips that store structured records. In operation, a caregiver can read the visible print directly, illuminate the light-reactive region to reveal emergency content, scan the code with an optical reader, or interrogate the data chip via contact pads or contactless interfaces (e.g., ISO/IEC 7816, ISO/IEC 14443) to retrieve versioned, access-controlled records. Optional features may include microprinting, tactile or Braille text, tamper-evident elements, and a reflective border to aid discovery. Alternatives to the card form factor may include but are not limited to a wristband, tag, key fob, and/or adhesive label, or mobile-wallet credential that uses equivalent modalities (printed text/images, light-reactive markings, optically encoded symbols, and electronic memory) to store and expose the patient's medical information.

Mobile reader 200 may be a handheld device configured for use by emergency services personnel to obtain patient medical information suitable for emergency care, with or without the patient being conscious. The reader incorporates a light emitter capable of producing visible and ultraviolet bands to illuminate light-reactive ink on patient card 100, an imaging sensor and decoder to interpret optically encoded symbols (e.g., QR/Aztec/1-D barcodes), and a contactless interface (e.g., NFC compliant with ISO/IEC 14443) to interrogate an on-card data chip that exposes an emergency-only dataset. In operation, the device activates the illumination source, captures an image of the reactive region or code, decodes the payload locally, and enforces a preconfigured emergency access policy that limits retrieval and display to a defined subset (e.g., allergies, contraindications, active conditions), optionally authenticating the reader by device credential before enabling chip access. A sealed enclosure, glove-operable controls, integrated display, rechargeable power source, and optional wired or wireless link to a processing system support field use, offline operation, and post-event audit logging. Alternatives include but are not limited to a smartphone or tablet running a reader application with an accessory illumination module, a wrist-mounted or chest-mounted scanner integrated into responder gear, a multi-function ambulance console with the same illumination, imaging, and contactless capabilities, or a compact clip-on optical reader that pairs with existing EMS devices to provide the described emergency-subset acquisition.

Stationary reader 300 may be a surface-mounted device (e.g. mounted to a countertop, tabletop, desktop, cabinet side, cabinet door, wall, etc.) configured to access, and in some embodiments modify, medical information stored on a data chip of patient card 100 for use in clinical settings. The reader interfaces with a healthcare computing device (e.g., workstation or EHR terminal) to deliver records and, when permitted, to accept updates from the host system. It implements a chip interface supporting contact (e.g., ISO/IEC 7816) and/or contactless (e.g., ISO/IEC 14443/NFC) protocols, a controller with secure firmware, and a policy module that requires user verification before exposing protected fields. Verification may be accomplished through credentials supplied via the host (username/password or single sign-on), a clinician smart card or proximity badge, or an integrated biometric sensor, with the reader establishing a cryptographic session to the card and enforcing role-based field access. Access attempts or events may be logged to the host for audit. Host connectivity may include USB, Ethernet, and/or other known methods in the art, and the device can operate in read-only or read/write modes based on policy. Alternatives include a reader integrated into a keyboard or monitor bezel, a kiosk or registration desk unit, a USB dongle form factor, or a multi-function clinical card terminal that provides the same chip access and user-verification functions.

Healthcare computer 102 may be a general-purpose computing device (e.g., workstation or clinical terminal) configured to pair with stationary reader 300 to access, display, and, when permitted by policy, modify medical data stored on patient card 100 and user records maintained in a database (e.g., database 104). In operation, computer 102 runs client software that communicates with reader 300 over a host interface (e.g., USB or Ethernet), establishes a cryptographic session to the card through the reader, authenticates the clinician via local or federated credentials, and enforces role-based access controls before enabling read/write transactions. The same software can query or update database 104 through secure APIs and record audit logs to a facility log store. Computer 102 may further interoperate with electronic health record systems and medical coding and billing platforms by mapping retrieved fields to standardized vocabularies (e.g., ICD, CPT/HCPCS) and exchanging messages using healthcare data formats (e.g., HL7/FHIR) or claims formats (e.g., X12), thereby supporting charge capture and claims preparation without re-entry. Security features can include TLS-protected transport, signed update packages, endpoint hardening, and least-privilege permissions. Alternatives include a thin-client terminal connected to a remote application server, a portable laptop or tablet running the same client functions, or a kiosk/registration workstation that performs the described reader pairing, database interaction, and coding/billing integration.

Processing system 103 may be a computing platform that executes the disclosed workflow by coordinating acquisition, policy-governed access, normalization, and/or persistence of patient medical information among patient card 100, mobile reader 200, stationary reader 300, healthcare computer 102, and medical database 104. In operation, processing system 103 may interface to the readers and host computers, establish cryptographically protected sessions, and restrict fields via redaction before presentation or storage.

Processing system 103 may further validate and normalize incoming data to standardized vocabularies and formats (e.g., ICD(International Classification of Diseases), CPT/HCPCS(Current Procedural Terminology/Healthcare Common Procedure Coding System), HL7/FHIR(Health Level Seven International's Fast Healthcare Interoperability Resources)), performs conflict resolution and version control for updates synchronized with the card chip and database, and records immutable audit entries with timestamps, device/user identifiers, and content hashes. A key-management module may maintain encryption materials for data in transit and at rest, and an alert or rules engine may generate contraindication or completeness flags for clinical systems.

Processing system 103 may be deployed remotely (e.g., cloud or data-center host), integrated on healthcare computer 102, or embedded within readers 200/300. Alternatives include a single-appliance gateway, a distributed microservices architecture, or an on-premises instance that provides the same interfaces, policy enforcement, synchronization, and audit functions.

Medical database 104 may be a secure, remote data store configured to maintain longitudinal patient medical records, including an emergency-access subset and metadata used for synchronization with patient card 100. The database serves requests from clinical systems and readers by enforcing role-and purpose-based access controls and exposing only those fields authorized for the requesting context. In operation, database 104 verifies possession of the patient card (e.g., a token derived from the card) and a user factor (passcode or biometric) before issuing a scope-limited session that permits read and, when policy allows, write operations. Records are stored in structured formats and may be normalized to healthcare data standards, with transport protected by TLS, data encrypted at rest under managed keys, immutable audit logging of access and modification events, and versioning to support reconciliation with chip-resident data. Alternatives include an on-premises facility database performing the same functions, a federated store backed by health-information exchange infrastructure, a cloud service that proxies to individual electronic health record systems, or a patient-controlled repository that synchronizes with the card while exposing clinician access under delegated authorization.

Network cloud 150 generally represents a network or collection of networks (such as the Internet or a corporate intranet, or a combination of both) over which the various components illustrated in FIG. 1 (including other components that may be necessary to execute the system described herein, as would be readily understood to a person of ordinary skill in the art). In particular embodiments, network 150 is an intranet, an extranet, a virtual private network (VPN), a local area network (LAN), a wireless LAN (WLAN), a wide area network (WAN), a metropolitan area network (MAN), a portion of the Internet, or another network 150 or a combination of two or more such networks 150. One or more links connect the systems and databases described herein to the network 150. In particular embodiments, one or more links each includes one or more wired, wireless, or optical links. In particular embodiments, one or more links each includes an intranet, an extranet, a VPN, a LAN, a WLAN, a WAN, a MAN, a portion of the Internet, or another link or a combination of two or more such links. The present disclosure contemplates any suitable network 150, and any suitable link for connecting the various systems and databases described herein.

The network 150 connects the various systems and computing devices described or referenced herein. In particular embodiments, network 150 is an intranet, an extranet, a virtual private network (VPN), a local area network (LAN), a wireless LAN (WLAN), a wide area network (WAN), a metropolitan area network (MAN), a portion of the Internet, or another network or a combination of two or more such networks 150. The present disclosure contemplates any suitable network 150.

One or more links couple one or more systems, engines or devices to the network 150. In particular embodiments, one or more links each includes one or more wired, wireless, or optical links. In particular embodiments, one or more links each includes an intranet, an extranet, a VPN, a LAN, a WLAN, a WAN, a MAN, a portion of the Internet, or another link or a combination of two or more such links. The present disclosure contemplates any suitable links coupling one or more systems, engines or devices to the network 150.

The interaction of this invention with other products or its environment is facilitated through distinct mechanisms. The mobile reader utilizes UV ink and light to read the UV code on the card, akin to scanning a bar code or QR code for quick access to information. In contrast, the stationary reader necessitates the physical insertion of the card in order to read the chip embedded within it. Furthermore, the stationary reader may enhance security measures by requiring a Personal Identification Number (PIN) from either the patient or a medical professional to access the sensitive information stored on the card. These interactions ensure secure and efficient access to patient medical data in both mobile and stationary healthcare settings.

### PATIENT IDENTITY CARD

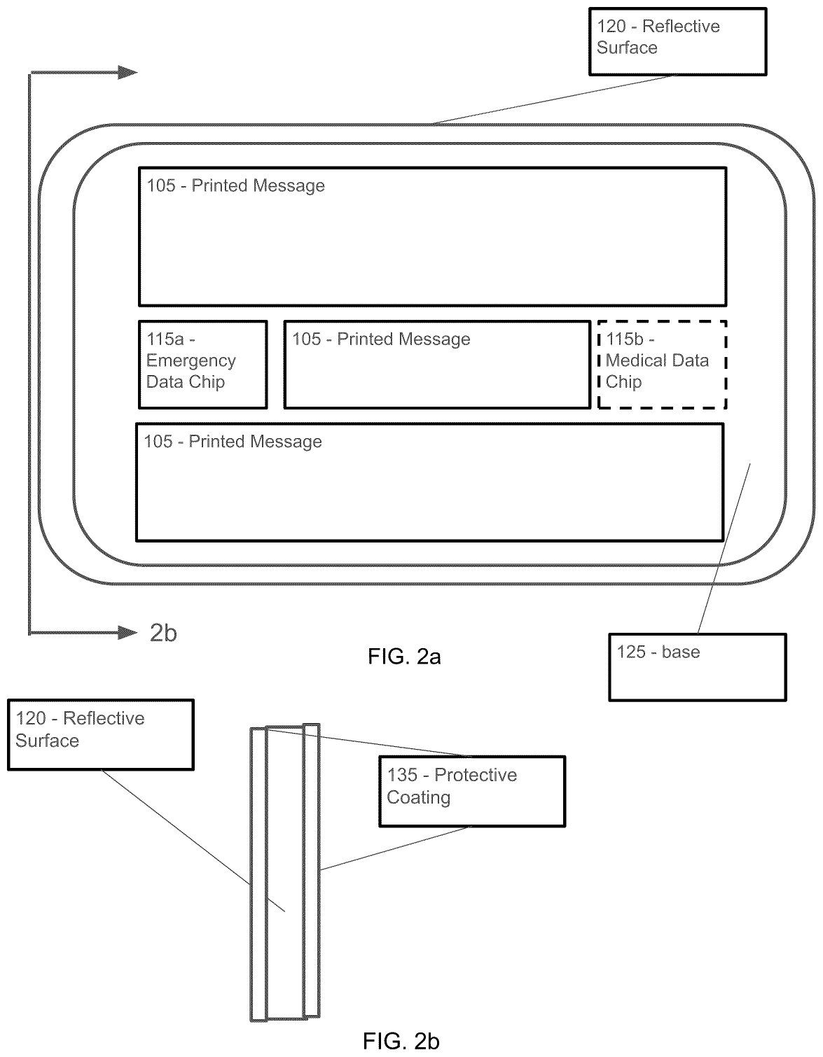

In reference to FIG. 2a-2c, the Patient Identity Card 100 may comprise a compact, credit card-sized device designed for easy portability by the patient. It serves as a repository for various types of data that contribute to patient identification and the transmission of medical information to authorized third parties. The Patient Identity Card 100 may comprise printed message 105, coded message 110, a data chip 115, a reflective surface 120, a base 125, a copying deterrent 130 and a protective coating 135.

Printed message 105 may be a region of visible indicia on one or both faces of patient card 100 that conveys non-protected information under normal ambient lighting to assist identification and card orientation. The message may comprise text and/or imagery such as the patient's name, a photograph or initial, a facility or plan identifier, standardized medical-alert iconography (e.g., allergy, anticoagulant use), and labels or legends identifying card features or use instructions (e.g., “Scan UV area,” “Tap for EMS”). Printed message 105 may use durable, high-contrast printing processes (e.g., offset lithography, dye-sublimation, thermal transfer, laser marking) with abrasion-and chemical-resistant pigments applied to or beneath a protective overcoat. Font sizes and pictograms are configured to remain legible in field conditions. The message deliberately excludes protected health information beyond consented identifiers, thereby reducing inadvertent disclosure while still directing responders to additional modalities on the card. Alternatives include molded-in or embossed characters, laser-etched graphics, applied labels or overlays, tactile or Braille markings for accessibility, or a replaceable front panel carrying equivalent non-protected indicia.

Coded message 110 may be a light-reactive printed region on one or both faces of patient card 100 that conveys emergency-relevant medical content in a form that is inconspicuous under ordinary lighting and readable under selected illumination (e.g., ultraviolet). In one embodiment, the coded message may be found on the opposite side of the card than the printed message 105. The coded message 110 functions to disclose sensitive but non-identifying clinical facts—such as allergy classes (e.g., beta-lactam, latex), anticoagulant status, implanted device category, or other contraindications—needed by first responders without exposing the information during routine handling. Implementation may include text or iconography printed with UV-fluorescent or UV-absorptive inks, and/or an optically scannable symbol (e.g., 1-D barcode, QR/Aztec) that encodes an emergency subset with error-correction, version indicators, and an optional checksum or digital signature. In practice, a mobile reader 200 illuminates the region, captures an image, and decodes the information subject to an emergency access policy. Print processes to produce the coded message 110 may include screen, flexographic, or laser-marking of the reactive layer beneath a protective overcoat to preserve spectral response. Alternatives include near-infrared or polarized-light-responsive markings, optically variable or holographic overlays that reveal content at specified viewing angles, microprinting or steganographic text readable with magnification, a visible-light barcode carrying the same emergency subset, or a peel-back/occluded label that physically conceals the information until intentional exposure.

Data chips 115 may comprise one or more integrated-circuit modules mounted to patient card 100 and configured to store and expose medical information to specialized readers. Each chip 115 may communicate via a standardized interface—contact pads positioned per ISO/IEC 7816-2 and/or a contactless near-field interface per ISO/IEC 14443—so that a reader can power, select, and exchange application protocol data units (APDUs) to retrieve or update defined records. The chips may implement secure messaging, access keys, and file structures with version counters and integrity tags to support controlled read/write operations and reconciliation with external systems. The data chips 115 may be located on the same face or on opposite faces of the card, and are positioned and oriented to provide reliable electrical or RF coupling to a chip reader. Alternatives include a single multi-application chip with logically partitioned emergency and medical domains, a contactless-only configuration, an embedded secure element laminated within the card body, or an optical/magnetic memory that provides equivalent data access under policy control.

Emergency data chip 115a may be a chip module, optionally included, dedicated to storing an emergency-access subset that mirrors or augments the information conveyed by coded message 110 (e.g., allergy classes, anticoagulant status, implanted device category, and other contraindications). In operation, a reader authenticates itself to the chip under an emergency policy and retrieves a constrained dataset intended for use when the patient may be unconscious, with the chip enforcing read-only or write-restricted access and returning records annotated with version and checksum fields. The chip 115a may be contact or contactless, placed to align with a reader slot or near-field antenna window, and may expose a minimal application identifier to simplify selection by emergency devices. Alternatives include omitting 115a and relying solely on coded message 110 for emergency content, hosting the emergency subset as a protected logical file on medical data chip 115b, or using a low-capacity passive tag that presents only signed, non-modifiable emergency fields.

Medical data chip 115b may be a chip module configured to store a larger set of patient information and/or to provide an access credential that enables a reader or healthcare computer to obtain sensitive records from medical database 104. The chip 115b implements mutual authentication and secure messaging, enforces role-and purpose-based permissions for read and write transactions, and maintains structured data with identifiers mapped to standardized vocabularies. When used as a pointer, the chip stores a token or certificate that, when presented with the patient card and a user factor, authorizes scoped retrieval and update through a secure network session. Placement affords proper mechanical insertion and RF coupling, and the chip 115b can be provisioned with lifecycle controls (activation, suspension, revocation) and audit metadata to support reconciliation with off-card records. Alternatives include storing only indexes and hashes with all detailed records fetched from database 104, implementing the medical dataset as a write-once archive with addenda, or consolidating all content into a single secure element with logical separation from the emergency subset.

Reflective surface 120 may be a printed or laminated optical layer disposed around the perimeter and carried over the edge of patient card 100, providing a high-visibility border discernible from both faces and from the card edge to aid discovery and orientation in low light without obstructing printed indicia or the placement and function of data chips 115. The surface operates by specular and/or retroreflective return of incident light and may be formed from Polyvinyl chloride(PVC), Poly(methyl methacrylate)(PMMA), Polycarbonates(PC), metallized pigments (e.g., aluminum-flake inks), a vacuum-metallized Polyethylene terephthalate(PET) foil, a microprismatic film (e.g., PMMA prisms), or glass-bead retroreflective media bound in a clear polymer, with the layer applied via hot-foil stamping, screen/flexographic printing, or adhesive lamination and sealed under a transparent protective overcoat. Keep-out zones and apertures are maintained so the reflective layer does not occlude optically scannable regions or interfere with chip 115 contact pads or near-field coupling. Alternatives include a dielectric mirror stack printed as an interference coating, a reflective thread or inlay embedded near the card edge, a holographic or optically variable border that remains non-opaque over functional areas, or a high-contrast non-metallic reflective ink that provides equivalent visibility while minimizing RF detuning of contactless interfaces.

Base 125 may be the structural substrate of patient card 100, providing mechanical support, dimensional stability, and a print-receptive surface for messages 105/110 while furnishing a mounting platform and routing features for data chips 115 (including 115a/115b). In one embodiment, base 125 is a multilayer laminate comprising a core sheet (e.g., PVC, Polyethylene Terephthalate Glycol(PETG), or polycarbonate) with adhesive tie layers and transparent overlaminates. The core may be surface-treated (e.g., corona or primer) to promote ink adhesion and may include formed cavities, lands, or through-slots that locate and protect chip modules while maintaining keep-out regions for optical codes and reader interfaces. The laminate stack resists abrasion and moisture, tolerates card personalization processes (printing, laser marking, embossing), and preserves RF transparency for contactless operation by avoiding continuous conductive layers in antenna regions. Local stiffeners or fillers may be used away from RF paths. The base 125 can be die-cut to finished geometry with chamfered or rounded edges and can accept the perimeter reflective surface without encroaching on printed fields or chip pads. Alternatives include a microporous polyolefin core with film overlam, a fiber-reinforced polymer composite for enhanced rigidity, a paperboard core sealed with polymer skins, a biodegradable PLA-based substrate for reduced environmental impact, or a metal-core construction with dielectric windows positioned to preserve near-field coupling while delivering equivalent structural and print-support functions.

Copying deterrent 130 may be a security feature on patient card 100 configured to indicate card legitimacy and discourage counterfeiting and unauthorized duplication. In one embodiment, deterrent 130 comprises an overt and/or covert printed element such as an optically variable device (e.g., holographic or diffractive foil with embedded microtext and guilloché patterns), color-shifting or UV-fluorescent inks, microprinting, latent images, and anti-scan halftone screens arranged so that genuine optical effects appear under prescribed lighting or viewing angles while replicas exhibit artifacts. The copying deterrent 130 may further include a serialized identifier laser-etched into the surface and cryptographically bound to a digital signature encoded in a 2D symbol elsewhere on the card, enabling machine verification by a compliant reader. Tamper-evident construction causes visible disruption if removal is attempted. Materials can include metallized PET foils, diffractive films, interference-pigment inks, thermochromic or photochromic chemistries, fluorescent phosphors, and taggant particles dispersed in a clear binder. Placement observes keep-out regions so as not to occlude messages 105/110 or interfere with data chips 115. Alternatives include an embedded security thread or watermark in base 125, a micro-optic (lenslet) film producing motion effects, forensic taggants detectable only under specialized instrumentation, or a purely electronic authenticity check using a chip-based challenge-response or physically unclonable function while retaining a minimal printed mark for human inspection.

Protective coating 135 may be a transparent, wear-resistant overlayer applied to patient card 100 to shield the printed message 105, coded message 110, reflective surface 120, and base 125 from abrasion, handling, moisture, and common cleaning agents, while preserving optical legibility (including UV readability of coded message 110) and maintaining RF performance for any contactless interfaces. In one embodiment, the coating is a polyurethane clear coat deposited over the card faces and edge and omitted at openings over the data chips 115 so as not to interfere with electrical contacts or near-field coupling. This layer forms a continuous polymer film that bonds to the card substrate via surface treatment or primer and cures to a hard, chemically stable finish. Application methods can include roll-coat, spray, curtain, or pad-coat processes followed by thermal or UV curing, with local masks or post-process ablation defining chip keep-outs. Alternatives include acrylic or epoxy clear coats, parylene conformal coatings, polysiloxane hardcoats, or laminated clear films (e.g., PET or polycarbonate overlaminates), with optional additives such as UV absorbers, anti-glare or matte agents, and antimicrobial compounds, provided they do not occlude optically scannable regions or detune contactless interfaces.

### MOBILE READER 200

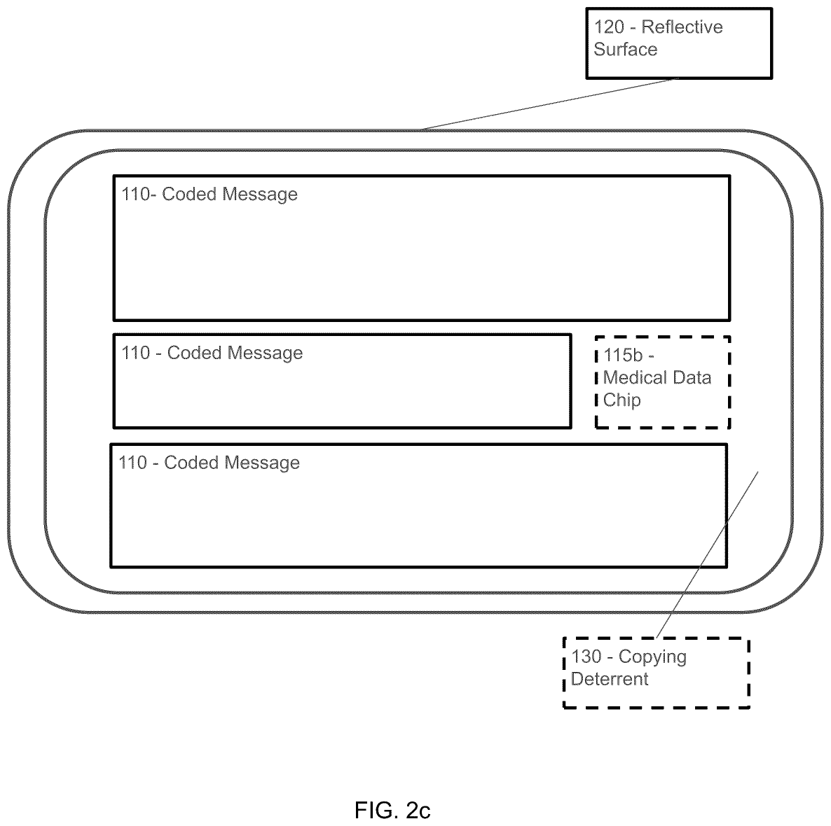

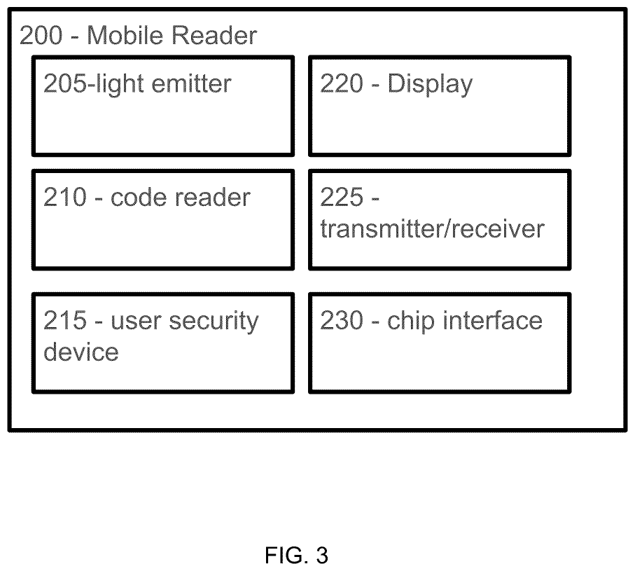

In reference to FIG. 3, the Mobile Reader 200 is a handheld device designed to interact with the Patient Identity Card 100. The Mobile reader 200 may comprise a light emitter 205, a code reader 210, a user security device 215, a display 220, and a transmitter/receiver 225.

Light emitter 205 may be an illumination module of mobile reader 200 configured to excite and render coded message 110 legible to the reader's imaging sensor and to human operators. In one embodiment, emitter 205 comprises one or more solid-state sources (e.g., UV-A LEDs centered at 365-405 nm and, optionally, visible white LEDs) mounted on a thermally managed substrate and driven by a constant-current driver to deliver uniform irradiance across the printed region. Integrated optics (e.g., diffuser, TIR lens, or light pipe) and a baffled shroud may be used to confine the beam and minimize specular glare from card 100. A UV-transmissive window (e.g., fused silica or UV-grade PMMA) protects the emitters, and an optical bandpass filter may be paired with the downstream sensor to improve contrast. Firmware may pulse or modulate the UV output to reduce power draw and to enable synchronous detection, and output levels can be limited to photobiological safety guidelines. Materials can include InGaN/AlGaN die, aluminum core PCB, and polycarbonate housing. Alternatives include a UV laser diode with a diffuser, a xenon or LED flash with UV component, a ring-light or coaxial illumination geometry, or a near-infrared emitter matched to inks responsive outside the visible spectrum while providing equivalent excitation of the coded message.

Code reader 210 is an optical sensing and decoding subsystem of mobile reader 200 configured to interpret the message on patient card 100 after illumination by light emitter 205. In one embodiment, reader 210 includes a solid-state image sensor (e.g., CMOS rolling-shutter array) with fixed-focus optics, an optical bandpass filter matched to the emission of the reactive inks, and a controller executing symbol-decoding and text recognition routines to recover payloads from 1-D and 2-D codes (e.g., Code 128, PDF417, QR, Aztec) and to enhance legibility of UV-revealed text or icons. Operation comprises capturing frames under controlled exposure synchronized to emitter 205, performing gain and distortion correction, localizing the printed region, and applying error-correction and signature checks before outputting decoded fields subject to emergency-access policy constraints. The subsystem may also provide confidence scores and store an audit thumbnail. Materials can include a glass or molded-plastic lens stack, coated protective window, polymer housing, and a microcontroller or FPGA with nonvolatile memory for firmware. Alternatives include a laser line-scanner with photodiode receiver for 1-D symbologies, a higher-sensitivity CCD camera, a polarization-selective or multispectral imager for glare reduction and ink contrast, or an accessory camera module paired with a host device that performs equivalent capture and decoding functions.

User security device 215 is an operator-authentication subsystem of mobile reader 200 configured to verify that a person is authorized to activate the reader and access emergency or clinical functions. In one embodiment, device 215 implements multi-factor verification by combining a knowledge factor (PIN entered on a sealed keypad or touchscreen), a possession factor (proximity badge or smart card read via an integrated NFC/ISO-14443 interface), and/or a biometric factor (fingerprint sensor or camera for facial verification), with a secure element storing device and verifier credentials and performing challenge-response without exposing secret material. On power-up or prior to sensitive operations, the reader gates UI and chip/code access until credentials are validated, establishes a role for the operator, and issues a scope-limited session token used by the local policy engine. Failed attempts are rate-limited, and events are logged for audit. Hardware may include a hardened microcontroller, capacitive or optical fingerprint module, RFID/NFC antenna, keypad or touch digitizer, secure element IC, and polymer or metal housing with tamper-evident features. Alternatives include single-factor verification (e.g., PIN only) for offline EMS use, federated sign-on via a paired healthcare computer, FIDO2/U2F security keys, Bluetooth-paired badge readers, or remote attestation against a facility identity service, each providing equivalent authorization gating before enabling reader functions.

Display 220 may be a user interface module of mobile reader 200 configured to present decoded emergency information and operational prompts and to accept operator input for navigation and acknowledgments. In one embodiment, display 220 is a sealed, impact-resistant touchscreen (e.g., transmissive LCD or OLED panel with projected-capacitive sensor) bonded to a chemically strengthened cover lens using optically clear adhesive, providing sufficient luminance for daylight readability and a dimmable “night” setting for low-light use. The touch controller supports gloved and wet-surface operation, and the firmware renders role-appropriate screens (e.g., allergy classes, contraindication flags, device status) while gating sensitive functions until user security device 215 validates the operator. The module interfaces with the reader's controller over a digital display bus, includes a hardware backlight driver (for LCD) or power management (for OLED), and may provide haptic or audible feedback to confirm selections. Gasketing and coatings (anti-reflective, oleophobic) protect against fluids and abrasion. Alternatives include a non-touch display with dedicated hard keys, a high-contrast e-paper or transflective panel for extended battery life, a segmented or icon display with limited prompts, or omission of the integral screen in favor of a paired host device (e.g., smartphone/tablet) that provides equivalent visual and input functions via a wired or wireless link.

Transmitter/receiver 225 may be an optional communications subsystem of mobile reader 200 configured to establish a secure network link to processing system 103 and, when permitted by policy, to medical database 104 for retrieval of emergency-appropriate records and post-event audit upload. In one embodiment, transmitter/receiver 225 includes one or more wireless interfaces (e.g., IEEE 802.11 Wi-Fi and cellular LTE/5G) with integrated or board-mounted antennas, RF front-end modules, baseband controller, and a secure networking stack that performs mutual authentication using device credentials stored in a secure element. Once associated, the reader transmits a scope-limited query derived from the patient card token or chip credential and receives a redacted dataset, which is cached locally for session use and logged for reconciliation. The module may support secondary links (e.g., Bluetooth for tethering) and wired backhaul through a sealed USB/Ethernet interface when docked, with transport protected by cryptographic protocols and certificate pinning to the facility endpoint. Hardware can include multilayer PCB with RF shielding cans, matching networks, SAW/BAW filters, ceramic or PIFA antennas, and conformal coating for environmental protection. Alternatives include omitting the radio and using a host healthcare computer 102 as a gateway, employing a cradle with Ethernet for periodic synchronization, limiting connectivity to short-range tethering only, or operating fully offline with store-and-forward upload when a trusted link becomes available.

Chip interface 230 may be a chip-reading subsystem of mobile reader 200 configured to power, select, and exchange data with the data chips 115 (including emergency chip 115a and medical chip 115b) mounted on patient card 100. In one embodiment, interface 230 provides both contact and contactless operation. Materials can include gold-plated pogo pins, a molded or stamped contact frame, multilayer FR-4 PCB with RF components, ferrite sheet, and a polymer guide to align the card. Alternatives include a contactless-only interface, a contact-only slot without RF, an external sled or cradle that provides the chip interface while the handheld performs illumination and display, or an optical/magnetic memory reader that accesses equivalent chip-resident information encoded in a different medium.

### STATIONARY READER 300

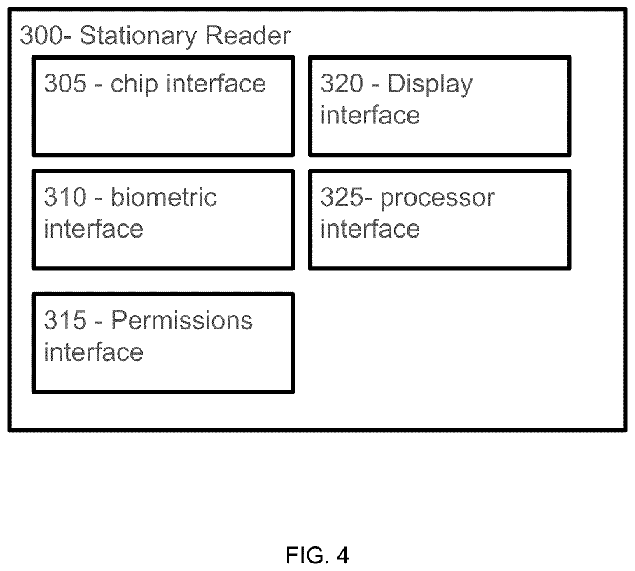

In reference to FIG. 4, the Stationary Reader 300 is a device designed to interface with a medical professional's computer 102 for the purpose of extracting patient information from the Patient Identity Card 100. The reader may incorporate encryption or other security measures to ensure the secure handling of sensitive medical data. The stationary reader 300 may comprise a chip interface 305, a biometric interface 310, a permissions interface315, a display interface320, and a processor interface 325 Chip interface 305 may be a card-access subsystem of stationary reader 300 configured to power, select, and exchange data with data chips 115 on patient card 100, including emergency chip 115a and medical chip 115b. In one embodiment, interface 305 provides dual-mode operation: a contact assembly and a contactless module comprising a tuned coil antenna. The Chip interface 305 aligns and retains the card using a keyed slot and guides, applies ESD protection and current-limited rails, performs application selection (AID), verifies integrity tags or digital signatures returned by the chip, and exposes records to the host healthcare computer 102 under reader policy. Mechanical and electrical components can include a molded contact frame, pogo-pin or leaf-spring contacts, multilayer FR-4 PCB with RF shielding cans, ferrite sheets, and a stainless or polymer chassis. Alternatives include a contact-only interface, a contactless-only antenna window, a swappable external sled providing the chip interface while the reader handles authentication and I/O, or an optical/magnetic memory reader that accesses equivalent on-card information encoded in a different medium.

Biometric interface 310 may be an operator-verification subsystem of stationary reader 300 configured to authenticate users before permitting access to or modification of data on medical database 104 and on data chips 115 of patient card 100. In one embodiment, interface 310 comprises a fingerprint sensor (capacitive or optical) integrated into the reader chassis and coupled to a secure element that enrolls and stores templates as non-invertible hashes. During use, the sensor captures a live sample, performs liveness detection (e.g., pulse, perspiration, sub-surface imaging), extracts features, and executes a match locally to derive an authentication assertion that the policy engine accepts to unlock scoped read/write operations. The module may alternatively or additionally include a face or iris camera with IR illumination and anti-spoofing, a palm-vein imager, or a voiceprint mic array, with image sensors protected by hardened glass and sealed gaskets for clinical cleaning. Communications between the sensor ASIC and secure processor are encrypted, and failed attempts are rate-limited and audit-logged. Materials can include molded polymer or stainless housings, chemically strengthened cover lenses, IR LEDs/VCSELs, and conformally coated PCBs. Alternatives include use of clinician smart cards or proximity badges as the primary factor with biometrics as a second factor, federated single sign-on via the paired healthcare computer 102 in lieu of on-device biometric matching, or an external, swappable biometric module certified to applicable healthcare security standards while providing equivalent permission gating.

Permissions interface 315 may be a credential-input subsystem of stationary reader 300 configured to receive and validate user authorization before enabling access to or modification of records on medical database 104 and data chips 115 of patient card 100. In one embodiment, interface 315 presents one or more input modalities—including a sealed keypad or touchscreen for PIN/username-password entry, a smart-card or proximity-badge reader (e.g., ISO/IEC 14443, FIDO2), and an optional one-time code prompt—whose outputs are conveyed to a secure processor that performs challenge—response against locally stored verifier keys or a facility identity service via the paired healthcare computer 102. Upon success, the reader derives a role and issues a scope-limited session token consumed by the policy engine to gate read/write APDUs and host API calls. The subsystem may include status indicators, tamper-detect circuitry, encrypted links to a secure element, and audit logging of authentication events with time, device, and role metadata. Hardware can comprise a hardened microcontroller, contactless antenna and matching network, keypad/touch digitizer, and polymer or metal fascia sealed for clinical cleaning. Alternatives include exclusive use of federated single sign-on relayed from computer 102, reliance on clinician smart cards as the sole factor with PIN-unlock, use of biometric interface 310 as a primary factor with PIN as backup, or a removable authentication sled providing equivalent credential capture and verification while the reader enforces permissions locally.

Display interface 320 may be a basic visual output module of stationary reader 300 configured to present device status, authentication prompts, access results, and error or audit notifications to an operator during interactions with patient card 100 and medical database 104. In one embodiment, the interface comprises a compact, sealed display—such as a mono or color LCD or OLED panel—driven by the reader's controller over a digital display bus, with a hardened cover lens bonded by optically clear adhesive and gasketed to withstand clinical cleaning. Firmware renders legible text and iconography indicating reader readiness, credential verification outcomes, chip interface 305 activity (e.g., read/write progress), and policy gating messages, and may provide simple soft-key labels if the reader includes adjacent buttons. The module can include an ambient light sensor for automatic brightness control and an audio or LED indicator for confirmatory cues. Materials can include a molded polymer or metal bezel, chemically strengthened glass or coated polycarbonate lens, conformally coated PCB, and EMI shielding as needed. Alternatives include a segmented or e-paper display for low power, a larger touchscreen shared with host controls, LED status clusters only, or omission of an integral screen when feedback is provided exclusively through the paired healthcare computer 102 while the reader maintains the same functional notifications over the host link.

Processor interface 325 may be a communications and control subsystem of stationary reader 300 configured to exchange commands, data records, and status with a processor local to the reader, with a processor of a paired healthcare computer 102, or with a remote processor hosted by processing system 103. In one embodiment, interface 325 exposes one or more physical transports (e.g., USB, Ethernet, UART, I²C/SPI) terminated on a secure microcontroller or SoC that implements framed messaging, flow control, and authenticated sessions. Firmware presents an application protocol for chip interface 305 transactions (e.g., APDU pass-through, read/write progress, integrity tags), authentication results from permissions interface 315 and biometric interface 310, and audit/event telemetry destined for medical database 104. The subsystem may include DMA-backed buffers for high-reliability transfers, interrupt lines for low-latency signaling, and cryptographic services (TLS with certificate pinning or MAC-based link security) anchored in a secure element. EMI shielding, ESD protection, and galvanic isolation can be provided to meet clinical safety and IT requirements. Materials can include multilayer FR-4 PCBs with shield cans, magnetics for Ethernet, USB transceivers, level shifters, and molded or metal I/O connectors sealed for cleaning. Alternatives include a wireless host link (Wi-Fi/Bluetooth) with the same authenticated protocol, a thin-client architecture wherein the reader exposes only a minimal register interface and all processing occurs on computer 102, or a message-broker middleware (e.g., gRPC/REST over TLS) terminating directly on a remote processor while preserving the described authenticated, scoped data exchange.

### THE PROCESSING SYSTEM 103

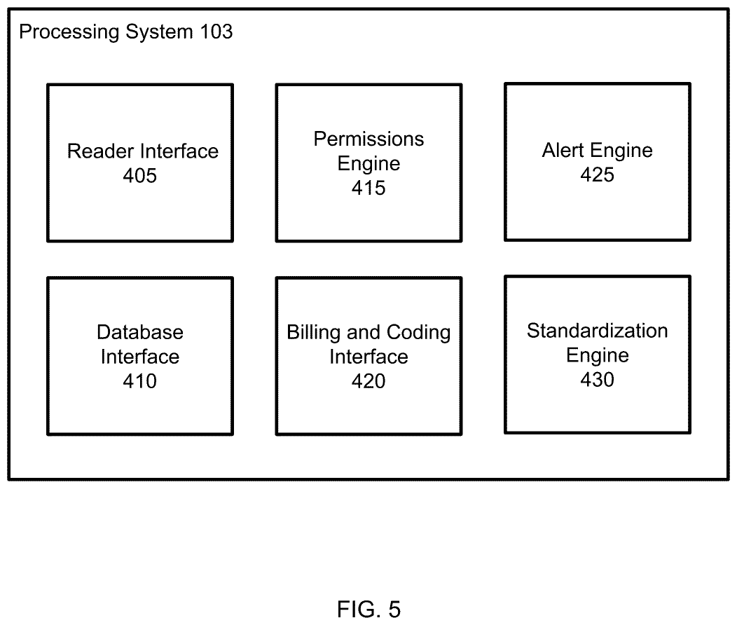

In reference to FIG. 5, the processing system 103 may comprise a reader interface(405), a database interface (410), a permissions engine (415), a billing and coding interface (420), alert engine(425), and a standardization engine(430).

Reader interface 405 may be a communications and ingestion module of processing system 103 configured to receive, authenticate, and normalize data originating from mobile reader 200 and stationary reader 300. In operation, interface 405 exposes authenticated endpoints (e.g., mutually authenticated TLS over REST/gRPC or message-queue topics) that accept payloads comprising decoded emergency subsets, chip transactions (APDU results, integrity tags, version counters), operator-authentication assertions, and device telemetry. A verifier anchored in a hardware-backed key store validates reader certificates or tokens, enforces role-and purpose-based scopes, applies schema and signature checks, and rate-limits or rejects malformed traffic. The module transforms inbound records to internal canonical models (e.g., HL7/FHIR mappings where applicable), annotates them with device/user/time metadata, and forwards them to downstream services such as policy resolution, audit logging, and database synchronization. Implementation may comprise containerized microservices running on general-purpose servers with load balancers, API gateways, and observability hooks (structured logs, metrics, traces). Alternatives include a lightweight socket protocol terminating on an on-premises gateway, a store-and-forward relay operating when readers are intermittently connected, or a direct peer-to-peer mode in which computer 102 proxies reader data while interface 405 retains the same authentication, normalization, and forwarding functions.

Database interface 410 may be a service layer of processing system 103 configured to send and receive patient-record information to and from medical database 104 under policy and integrity constraints. In operation, interface 410 establishes mutually authenticated, encrypted sessions (e.g., TLS with client certificates or token-based authorization), exposes read/write APIs mapped to canonical schemas, and executes transactional operations that include field-level validation, version checks, and integrity-tag verification before committing updates Responses are normalized to internal models and annotated with provenance (device, operator, time) for downstream audit and synchronization with card-resident data. Implementation may comprise an ORM-backed data access module, stored-procedure adapters, and message handlers (e.g., REST/gRPC or queue consumers) running on general-purpose servers or containers, with connection pooling, retry/backoff, and circuit-breaker controls for resilience. Cryptographic keys are maintained in a hardware-backed store. Alternatives include a direct database driver with least-privilege roles and row-level security, a middleware connector to a health-information exchange or EHR system using HL7/FHIR or X12 transactions, or a gateway that proxies requests to multiple federated repositories while preserving the same authenticated, scoped, and versioned data exchange semantics.

Permissions engine 415 may be an authorization subsystem of processing system 103 configured to determine whether a requesting operator and device may access specified patient information and to constrain the scope of any permitted transaction. In operation, engine 415 receives authentication assertions and context from upstream components (e.g., reader certificates, user credential or biometric results, patient-consent flags, purpose-of-use, location, and emergency/non-emergency mode), evaluates those attributes against a policy set, and returns a decision with obligations that may include field-level redaction, operation limits (read-only vs. write), and time-bounded scope tokens. The engine may implement role-based and attribute-based controls (RBAC/ABAC) using a policy decision point (PDP) and policy enforcement points (PEPs) at API boundaries, with policies expressed in a machine-readable language and versioned, signed, and stored in a secure repository. Decisions are logged with inputs and outcomes for audit, and cryptographic keys used to sign tokens are kept in a hardware-backed store. Alternatives include delegating authorization to an external identity and access management service (e.g., federated SSO/OPA), embedding minimal policy checks on the reader or healthcare computer with the central engine providing token minting only, or using capability-based credentials that encode permitted fields and operations without separate online evaluation.

Billing and coding interface 420 may be a service layer of processing system 103 configured to interoperate with medical billing and coding platforms so that updates associated with use of patient identity card 100 are translated into standardized charge and diagnosis artifacts. In operation, interface 420 ingests encounter data and clinician annotations received via readers and healthcare computer 102, normalizes fields to canonical clinical models, and maps them to codified representations (e.g., ICD diagnosis codes, CPT/HCPCS procedure codes, NDC where applicable), applying configurable rules for code selection, modifiers, and medical necessity checks. The module then packages results into compliant transactions (e.g., HL7/FHIR resources, X12837/835, or proprietary vendor APIs), digitally signs or otherwise attests to provenance, and returns acknowledgments and error details for reconciliation. Implementation may include terminology services, rule engines, validation pipelines, and connectors with retry/backoff and audit logging, executing as containerized microservices or library components within the processing stack. Alternatives include a pass-through adapter that forwards normalized data to a third-party coding service for code assignment, a batch export that produces codified artifacts for later submission by a revenue-cycle system, or a minimal interface that emits only structured FHIR resources while external billing platforms perform code mapping and claims assembly.

Alert engine 425 may be a clinical-decision subsystem of processing system 103 configured to evaluate patient medical history from medical database 104 and card-resident records and to generate alerts indicating potential drug-drug, drug-allergy, and drug-condition interactions, contraindications, duplicative therapies, and dosage issues. In operation, engine 425 ingests structured inputs (e.g., active medications, allergies, problem list, vitals, renal/hepatic indicators) normalized to standard vocabularies (e.g., RxNorm, SNOMED CT, LOINC), resolves code systems, and executes rules and reference checks against a curated knowledge base. The module tiers results by severity and clinical relevance, suppresses redundant findings using de-duplication and context filters, and emits machine-readable alert objects with rationale, citations, and suggested actions for display on readers and healthcare computer 102. Implementation may include a rules interpreter, constraint solver, and optional statistical or ML scorers, backed by versioned drug-interaction tables and contraindication lists, with decisions signed and audit-logged along with input snapshots for later review. Transport occurs via authenticated APIs and outputs can be embedded into FHIR resources. Alternatives include delegating all interaction checking to an external clinical decision support service via a standards-based API, hosting a lightweight on-device emergency checker on mobile reader 200 for offline use with periodic synchronization, or limiting the subsystem to pass-through of third-party adjudication results while preserving the same alert packaging, provenance, and audit semantics.

Standardization engine 430 may be a data-normalization and schema-mapping subsystem of processing system 103 configured to transform heterogeneous inputs—originating from readers 200/300, healthcare computer 102, and external systems—into canonical representations suitable for storage on patient card 100 and medical database 104. In operation, engine 430 validates incoming payloads against structural and semantic constraints, reconciles vocabularies and code systems (e.g., mapping local terms to SNOMED CT, RxNorm, LOINC, and ICD/CPT/HCPCS), harmonizes units and measurement conventions, resolves identifiers, and converts messages to target schemas (e.g., TLV/record layouts for chip-resident files and HL7/FHIR resources for database persistence), recording version and provenance metadata for audit and rollback. The subsystem may implement terminology services, deterministic transformation rules, and configurable crosswalk tables, with integrity checks (hashes/signatures) and field-level redaction applied before emission. Failed validations yield structured error reports for correction. Implementation can comprise containerized services or libraries executing on general-purpose servers with access to a secured terminology store. Alternatives include delegating normalization to an external interoperability gateway, performing minimal on-the-fly coercion on the readers with centralized reconciliation deferred to the database layer, or using capability-specific adapters that emit pre-standardized artifacts while engine 430 limits its role to verification and pass-through.

Referring now to FIG. 4, there is shown a block diagram depicting an exemplary computing device 10 suitable for implementing at least a portion of the features or functionalities disclosed herein. Computing device 10 may be, for example, any one of the computing machines listed in the previous paragraph, or indeed any other electronic device capable of executing software-or hardware-based instructions according to one or more programs stored in memory. Computing device 10 may be configured to communicate with a plurality of other computing devices, such as clients or servers, over communications networks such as a wide area network a metropolitan area network, a local area network, a wireless network, the Internet, or any other network, using known protocols for such communication, whether wireless or wired.

In one aspect, computing device 10 includes one or more central processing units (CPU) 12, one or more interfaces 15, and one or more busses 14 (such as a peripheral component interconnect (PCI) bus). When acting under the control of appropriate software or firmware, CPU 12 may be responsible for implementing specific functions associated with the functions of a specifically configured computing device or machine. For example, in at least one aspect, a computing device 10 may be configured or designed to function as a server system utilizing CPU 12, local memory 11 and/or remote memory 16, and interface(s) 15. In at least one aspect, CPU 12 may be caused to perform one or more of the different types of functions and/or operations under the control of software modules or components, which for example, may include an operating system and any appropriate applications software, drivers, and the like.

CPU 12 may include one or more processors 13 such as, for example, a processor from one of the Intel, ARM, Qualcomm, and AMD families of microprocessors. In some embodiments, processors 13 may include specially designed hardware such as application-specific integrated circuits (ASICs), electrically erasable programmable read-only memories (EEPROMs), field-programmable gate arrays (FPGAs), and so forth, for controlling operations of computing device 10. In a particular aspect, a local memory 11 (such as non-volatile random-access memory (RAM) and/or read-only memory (ROM), including for example one or more levels of cached memory) may also form part of CPU 12. However, there are many different ways in which memory may be coupled to system 10. Memory 11 may be used for a variety of purposes such as, for example, caching and/or storing data, programming instructions, and the like. It should be further appreciated that CPU 12 may be one of a variety of system-on-a-chip (SOC) type hardware that may include additional hardware such as memory or graphics processing chips, such as a QUALCOMM SNAPDRAGON™ or SAMSUNG EXYNOS™ CPU as are becoming increasingly common in the art, such as for use in mobile devices or integrated devices.

As used herein, the term “processor” is not limited merely to those integrated circuits referred to in the art as a processor, a mobile processor, or a microprocessor, but broadly refers to a microcontroller, a microcomputer, a programmable logic controller, an application-specific integrated circuit, and any other programmable circuit.

In one aspect, interfaces 15 are provided as network interface cards (NICs). Generally, NICs control the sending and receiving of data packets over a computer network; other types of interfaces 15 may for example support other peripherals used with computing device 10. Among the interfaces that may be provided are Ethernet interfaces, frame relay interfaces, cable interfaces, DSL interfaces, token ring interfaces, graphics interfaces, and the like. In addition, various types of interfaces may be provided such as, for example, universal serial bus (USB), Serial, Ethernet, FIREWIRE™, THUNDERBOLT™, PCI, parallel, radio frequency (RF), BLUETOOTH™, near-field communications (e.g., using near-field magnetics), 802.11 (WiFi), frame relay, TCP/IP, ISDN, fast Ethernet interfaces, Gigabit Ethernet interfaces, Serial ATA (SATA) or external SATA (ESATA) interfaces, high-definition multimedia interface (HDMI), digital visual interface (DVI), analog or digital audio interfaces, asynchronous transfer mode (ATM) interfaces, high-speed serial interface (HSSI) interfaces, Point of Sale (POS) interfaces, fiber data distributed interfaces (FDDIs), and the like. Generally, such interfaces 15 may include physical ports appropriate for communication with appropriate media. In some cases, they may also include an independent processor (such as a dedicated audio or video processor, as is common in the art for high-fidelity A/V hardware interfaces) and, in some instances, volatile and/or non-volatile memory (e.g., RAM).

Although the system shown in FIG. 4 illustrates one specific architecture for a computing device 10 for implementing one or more of the embodiments described herein, it is by no means the only device architecture on which at least a portion of the features and techniques described herein may be implemented. For example, architectures having one or any number of processors 13 may be used, and such processors 13 may be present in a single device or distributed among any number of devices. In one aspect, single processor 13 handles communications as well as routing computations, while in other embodiments a separate dedicated communications processor may be provided. In various embodiments, different types of features or functionalities may be implemented in a system according to the aspect that includes a client device (such as a tablet device or smartphone running client software) and server systems (such as a server system described in more detail below).

Regardless of network device configuration, the system of an aspect may employ one or more memories or memory modules (such as, for example, remote memory block 16 and local memory 11) configured to store data, program instructions for the general-purpose network operations, or other information relating to the functionality of the embodiments described herein (or any combinations of the above). Program instructions may control execution of or comprise an operating system and/or one or more applications, for example. Memory 16 or memories 11, 16 may also be configured to store data structures, configuration data, encryption data, historical system operations information, or any other specific or generic non-program information described herein.

Because such information and program instructions may be employed to implement one or more systems or methods described herein, at least some network device embodiments may include nontransitory machine-readable storage media, which, for example, may be configured or designed to store program instructions, state information, and the like for performing various operations described herein. Examples of such nontransitory machine-readable storage media include, but are not limited to, magnetic media such as hard disks, floppy disks, and magnetic tape; optical media such as CD-ROM disks; magneto-optical media such as optical disks, and hardware devices that are specially configured to store and perform program instructions, such as read-only memory devices (ROM), flash memory (as is common in mobile devices and integrated systems), solid state drives (SSD) and “hybrid SSD” storage drives that may combine physical components of solid state and hard disk drives in a single hardware device (as are becoming increasingly common in the art with regard to personal computers), memristor memory, random access memory (RAM), and the like. It should be appreciated that such storage means may be integral and non-removable (such as RAM hardware modules that may be soldered onto a motherboard or otherwise integrated into an electronic device), or they may be removable such as swappable flash memory modules (such as “thumb drives” or other removable media designed for rapidly exchanging physical storage devices), “hot-swappable” hard disk drives or solid state drives, removable optical storage discs, or other such removable media, and that such integral and removable storage media may be utilized interchangeably. Examples of program instructions include both object code, such as may be produced by a compiler, machine code, such as may be produced by an assembler or a linker, byte code, such as may be generated by for example a JAVA™ compiler and may be executed using a Java virtual machine or equivalent, or files containing higher level code that may be executed by the computer using an interpreter (for example, scripts written in Python, Perl, Ruby, Groovy, or any other scripting language).

In some embodiments, systems may be implemented on a standalone computing system. Referring now to FIG. 5, there is shown a block diagram depicting a typical exemplary architecture of one or more embodiments or components thereof on a standalone computing system. Computing device 20 includes processors 21 that may run software that carry out one or more functions or applications of embodiments, such as for example a client application. Processors 21 may carry out computing instructions under control of an operating system 22 such as, for example, a version of MICROSOFT WINDOWS™ operating system, APPLE macOS™ or iOS™ operating systems, some variety of the Linux operating system, ANDROID™ operating system, or the like. In many cases, one or more shared services 23 may be operable in system 20, and may be useful for providing common services to client applications. Services 23 may for example be WINDOWS™ services, user-space common services in a Linux environment, or any other type of common service architecture used with operating system 21. Input devices 28 may be of any type suitable for receiving user input, including for example a keyboard, touchscreen, microphone (for example, for voice input), mouse, touchpad, trackball, or any combination thereof. Output devices 27 may be of any type suitable for providing output to one or more users, whether remote or local to system 20, and may include for example one or more screens for visual output, speakers, printers, or any combination thereof. Memory 25 may be random-access memory having any structure and architecture known in the art, for use by processors 21, for example to run software. Storage devices 26 may be any magnetic, optical, mechanical, memristor, or electrical storage device for storage of data in digital form (such as those described above, referring to FIG. 4). Examples of storage devices 26 include flash memory, magnetic hard drive, CD-ROM, and/or the like.

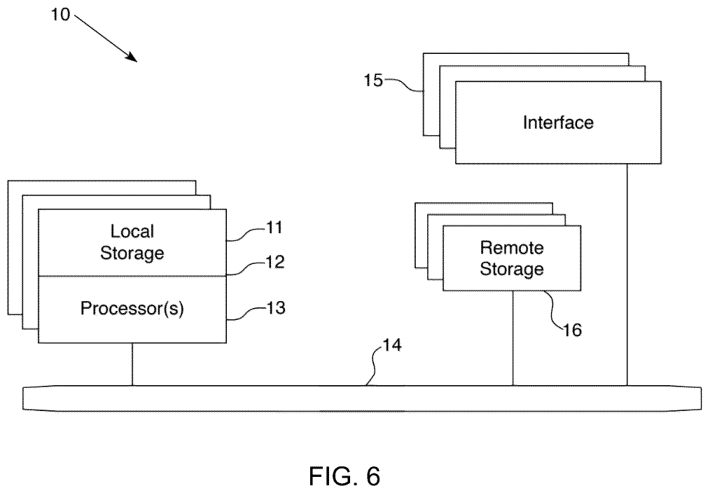

In some embodiments, systems may be implemented on a distributed computing network, such as one having any number of clients and/or servers. Referring now to FIG. 6, there is shown a block diagram depicting an exemplary architecture 30 for implementing at least a portion of a system according to one aspect on a distributed computing network.

According to the aspect, any number of clients 33 may be provided. Each client 33 may run software for implementing client-side portions of a system; clients may comprise a system 20 such as that illustrated in FIG. 5. In addition, any number of servers 32 may be provided for handling requests received from one or more clients 33. Clients 33 and servers 32 may communicate with one another via one or more electronic networks 31, which may be in various embodiments any of the Internet, a wide area network, a mobile telephony network (such as CDMA or GSM cellular networks), a wireless network (such as WiFi, WiMAX, LTE, and so forth), or a local area network (or indeed any network topology known in the art; the aspect does not prefer any one network topology over any other). Networks 31 may be implemented using any known network protocols, including for example wired and/or wireless protocols.

In addition, in some embodiments, servers 32 may call external services 37 when needed to obtain additional information, or to refer to additional data concerning a particular call. Communications with external services 37 may take place, for example, via one or more networks 31. In various embodiments, external services 37 may comprise web-enabled services or functionality related to or installed on the hardware device itself. For example, in one aspect where client applications are implemented on a smartphone or other electronic device, client applications may obtain information stored in a server system 32 in the cloud or on an external service 37 deployed on one or more of a particular enterprise's or user's premises.

In some embodiments, clients 33 or servers 32 (or both) may make use of one or more specialized services or appliances that may be deployed locally or remotely across one or more networks 31. For example, one or more databases 34 may be used or referred to by one or more embodiments. It should be understood by one having ordinary skill in the art that databases 34 may be arranged in a wide variety of architectures and using a wide variety of data access and manipulation means. For example, in various embodiments one or more databases 34 may comprise a relational database system using a structured query language (SQL), while others may comprise an alternative data storage technology such as those referred to in the art as “NoSQL” (for example, HADOOP CASSANDRA™, GOOGLE BIGTABLE™, and so forth). In some embodiments, variant database architectures such as column-oriented databases, in-memory databases, clustered databases, distributed databases, or even flat file data repositories may be used according to the aspect. It will be appreciated by one having ordinary skill in the art that any combination of known or future database technologies may be used as appropriate, unless a specific database technology or a specific arrangement of components is specified for a particular aspect described herein. Moreover, it should be appreciated that the term “database” as used herein may refer to a physical database machine, a cluster of machines acting as a single database system, or a logical database within an overall database management system. Unless a specific meaning is specified for a given use of the term “database”, it should be construed to mean any of these senses of the word, all of which are understood as a plain meaning of the term “database” by those having ordinary skill in the art.

Similarly, some embodiments may make use of one or more security systems 36 and configuration systems 35. Security and configuration management are common information technology (IT) and web functions, and some amount of each are generally associated with any IT or web systems. It should be understood by one having ordinary skill in the art that any configuration or security subsystems known in the art now or in the future may be used in conjunction with embodiments without limitation, unless a specific security 36 or configuration system 35 or approach is specifically required by the description of any specific aspect.

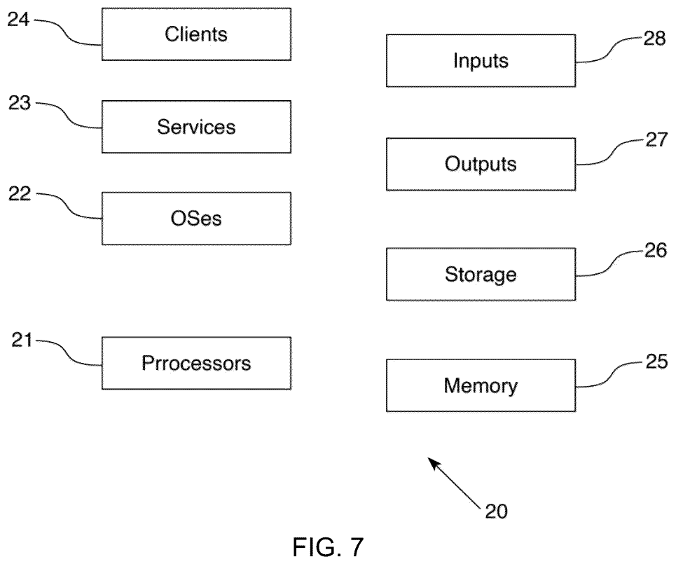

FIG. 7 shows an exemplary overview of a computer system 40 as may be used in any of the various locations throughout the system. It is exemplary of any computer that may execute code to process data. Various modifications and changes may be made to computer system 40 without departing from the broader scope of the system and method disclosed herein. Central processor unit (CPU) 41 is connected to bus 42, to which bus is also connected memory 43, nonvolatile memory 44, display 47, input/output (I/O) unit 48, and network interface card (NIC) 53. I/O unit 48 may, typically, be connected to keyboard 49, pointing device 50, hard disk 52, and real-time clock 51. NIC 53 connects to network 54, which may be the Internet or a local network, which local network may or may not have connections to the Internet. Also shown as part of system 40 is power supply unit 45 connected, in this example, to a main alternating current (AC) supply 46. Not shown are batteries that could be present, and many other devices and modifications that are well known but are not applicable to the specific novel functions of the current system and method disclosed herein. It should be appreciated that some or all components illustrated may be combined, such as in various integrated applications, for example Qualcomm or Samsung system-on-a-chip (SOC) devices, or whenever it may be appropriate to combine multiple capabilities or functions into a single hardware device (for instance, in mobile devices such as smartphones, video game consoles, in-vehicle computer systems such as navigation or multimedia systems in automobiles, or other integrated hardware devices).

In various embodiments, functionality for implementing systems or methods of various embodiments may be distributed among any number of client and/or server components. For example, various software modules may be implemented for performing various functions in connection with the system of any particular aspect, and such modules may be variously implemented to run on server and/or client components.

The skilled person will be aware of a range of possible modifications of the various embodiments described above. Accordingly, the present invention is defined by the claims and their equivalents.

### Additional Considerations

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

Some embodiments may be described using the expression “coupled” and “connected” along with their derivatives. For example, some embodiments may be described using the term “coupled” to indicate that two or more elements are in direct physical or electrical contact. The term “coupled,” however, may also mean that two or more elements are not in direct contact with each other, but yet still co-operate or interact with each other. The embodiments are not limited in this context.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and Bis false (or not present), A is false (or not present) and Bis true (or present), and both A and B are true (or present).

In addition, use of the “a” or “an” are employed to describe elements and components of the embodiments herein. This is done merely for convenience and to give a general sense of the invention. This description should be read to include one or at least one and the singular also includes the plural unless it is obvious that it is meant otherwise.

Upon reading this disclosure, those of skill in the art will appreciate still additional alternative structural and functional designs for a system and a process for creating an interactive message through the disclosed principles herein. Thus, while particular embodiments and applications have been illustrated and described, it is to be understood that the disclosed embodiments are not limited to the precise construction and components disclosed herein. Various apparent modifications, changes and variations may be made in the arrangement, operation and details of the method and apparatus disclosed herein without departing from the spirit and scope defined in the appended claims.

What is claimed is:

1. A rectangular card capable of fitting in a wallet for managing patient medical information, comprising:
a base layer having a length, width and thickness, the base layer comprising a first and second face, the faces defined by the length and width of the base layer and located on opposite sides of the card, and an edge surrounding the entirety of the perimeter of the base layer and defined by the thickness and either the length or width;
a first printed area located on the first face;
a second printed area located on the second face, the second printed area comprising UV-responsive ink printed on at least a portion of the second printed area, the UV-responsive ink encoding medical information readable under ultraviolet light;
an emergency data chip fixedly embedded in the base layer and accessible by an electronic reader from the first face;
a records data chip fixedly embedded in the base layer and accessible by an electronic reader from the first face or second face; and
a reflective border located on the edge of the base layer and extending uninterrupted partially onto both faces.

2. The rectangular card according to claim 1, wherein the records data is accessible from only the first face.

3. The rectangular card according to claim 1, wherein the records data is accessible from only the second face.

4. The rectangular card according to claim 1, further comprising a plurality of data labels, the data labels located substantially near and on the same face as the emergency data chip and records data chip, the data labels comprising text identifying the type of data held on the data chip.

5. The rectangular card according to claim 1, wherein the first printed area comprises text for identifying the card or card user.

6. The rectangular card according to claim 1, wherein the first printed area comprises icons and/or images representing medical alert information.

7. The rectangular card according to claim 1, wherein the second printed area comprises text comprising non-protected health information.

8. The rectangular card according to claim 1, wherein the second printed area comprises a code capable of interpretation by an electronic reader, but not by an individual.

9. The rectangular card according to claim 8, wherein the code is a QR code.

10. The rectangular card according to claim 1, wherein the records data chip is capable of storing data associated/representing an access code to provide permission to access a patient's medical history.

11. The rectangular card according to claim 1, wherein the records data chip is capable of storing data comprising a patient's medical history.

12. The rectangular card according to claim 1, wherein the records data and/or emergency data is encrypted.

13. The rectangular card according to claim 1, further comprising a protective coating applied over the card, except over the data chips.

14. The rectangular card according to claim 1, wherein the reflective border comprises PET, PVC, PMMA, and/or PC.

15. The rectangular card according to claim 1, wherein the reflective border is visible when viewed from the edge of the card, when the faces are obstructed.

16. The rectangular card according to claim 1, wherein the reflective border does not extend into the first or second printed areas.

17. The rectangular card according to claim 1, wherein the length is a range of 3 to 3.5 inches, the width is a range of 2 to 2.25 inches, and the thickness is a range of 0.03 to 0.10 inches.

18. The rectangular card according to claim 1, further comprising a protective coating wherein the protective coating comprises Polyurethane.

19. The rectangular card according to claim 18, wherein the protective coating covers all surfaces of the card, except at the interface of the records data chip and emergency data chip.
